(19) United States
(12) Patent Application Publication
(10) Pub. No.: US 2002/0146684 A1
(43) Pub. Date: Oct. 10, 2002

(54) One dimensional unichemo protection (UCP) in organic synthesis

(76) Inventors: Morten Peter Meldal, Frederiksberg (DK); Leslie Philip Miranda, Frederiksberg (DK)

Correspondence Address: MERCHANT & GOULD PC, P.O. BOX 2903, MINNEAPOLIS, MN, 55402-0903, US

(21) Appl. No.: 09/829,372

(22) Filed: Apr. 9, 2001

(51) Int. Cl. C12Q 1/70; C12Q 1/68; C07H 21/04; G01N 33/543

(52) U.S. Cl. 435/5; 435/6; 436/518; 530/320; 536/24.3

(57) ABSTRACT

A protected template molecule and a new one-dimensional UniChemo Protection (UCP) organic synthetic method for preparing polyfunctional organic molecules is described. The synthetic method can be used with many kinds of chemical reactions and provides selective access to many functional groups in a template molecule. The method utilizes protection groups that are each composed of building block units that can be removed one by one affording a new protection group one unit shorter or exposing a functional group on the template molecule. The exposed functional group on the template molecule can react with a target group. Different target groups can be introduced into the template molecule by using protection groups containing different numbers of building block units.

### FIELD OF THE INVENTION

The invention relates generally to orthogonal protection in organic synthesis. In particular, the invention relates to one-dimensional UniChemo Protection (UCP), a novel method of organic synthesis designed to provide selective access to multiple functional groups of a template molecule.

### BACKGROUND OF THE INVENTION

Orthogonal protection has been in use for many years in organic synthesis for preparing compounds with multiple functional groups. Typically, if two functional groups can react with a target group, one of the functional groups is blocked or protected from reaction leaving one group free to react with the target group. The protection group is then removed and the second functional group can react with a different target group. If there are more than two functional groups, multiple protection groups are required. The protection groups must be chosen such that they are compatible with each other and can be selectively removed.

An orthogonal protection system usually requires a set of completely independent protection groups. In a system of this kind, each protection group can be removed in any order, and in the presence of all other protection groups or functionality. Modulated lability strategies can provide graduations of chemical conditions, such as acidity, for selectivity. In general, there is a quadratic-like increase in the number of compatibility requirements with an increase in the number of functional groups using existing orthogonal protection strategies. This adds great complexity to the synthesis.

One variation of the orthogonal protecting groups uses halobenzyl ethers with a range of reactivity towards palladium (Pd) catalyzed amination followed by partially selective release of the benzyl group (see O. J. Plante, S. L. Buchwald, and P. H. Seeberger, J. Am. Chem. Soc., 122, p. 7148. (2001)). This approach is a special variation of the so-called safety catch principle, where a two-stage reaction sequence adds to the range of orthogonality that can be obtained.

One problem associated with the current methods for orthogonal protection of multiple functional groups is the compatibility requirements. Removal of a protection group requires unique chemical reactions that do not affect the other protection groups on the intermediate molecules or the template molecule itself. While selective reactions can be performed with a few orthogonally protected functional groups, the design of a larger synthetic scheme becomes extremely complex and, in some instances, impossible.

Another problem with existing synthetic methods using orthogonal protection groups is the wide variation in the chemical reaction conditions used for introduction and cleavage of the various protection groups. This often precludes automation of the reactions and obtaining high yield of the various target groups. Therefore, such methods are typically not suitable for high throughput synthesis.

Another problem with the existing synthetic method using orthogonal protection is the large number of steps required to prepare a target molecule with even a few different functional groups. Low yields are typical for the target molecules.

### SUMMARY OF THE INVENTION

The invention provides methods of preparing a target compound with different functionality. In one embodiment, a target compound with different functionality can be formed by initially preparing two or more protection groups comprising building block units linked together. A protected compound is formed containing two or more protective groups. At least two of the protection groups contain a different number of building block units. A terminal building block unit is removed from each protection group using one or more chemical, electrochemical, or photolytic reactions. Additional building blocks are consecutively removed from each building block unit. As each protection group is completely removed, the newly formed intermediate compound can react with a target group.

The present invention also provides a protected template molecule and a new one-dimensional UniChemo Protection (UCP) organic synthetic method for preparing polyfunctional organic molecules. A UniChemo Protected compound is formed by reacting a template molecule and various protection groups. The template molecule has more than one functional group. The protection groups are attached to the template molecule through the functional groups of the template molecule. The protection groups comprise building block units linked together. Each protection group contains one active building block group and can contain one or more inert building block units. At least two of the protection groups contain different numbers of building blocks. In one embodiment, each protection group contains a different number of building block units. The UCP compound can contain up to 1000 protection groups.

Chemical, electrochemical, or photolytic reactions are used to remove the active building block unit from each protection group. For protection groups containing at least two building block units, the removal of the active group results in the formation of a shorter protection group by one building block unit. Alternatively, the removal of the active group from a protection group containing only one building block unit results in the complete removal of the protection group from a functional group of the template molecule. The removal of a protection group from a functional group of the template molecule results in an exposed functional group in the template molecule that can react with a desired target group. Only functional groups of the template molecule without a protection group can react with the desired target group. The desired target group and the protection groups are chosen such that the target group does not react with the protection groups.

Additional building block units are consecutively removed from the remaining protection groups using chemical, electrochemical, or photolytic reactions to form even shorter protection groups and at least one additional exposed functional group of the template molecule that is not attached to a protection group. The newly exposed functional group is then reacted with another desired target group. The second target group added is generally different from the first target group. The process of removing one building block unit of the protection group can be repeated and followed by the reaction of any exposed functional group of the template molecule with another target group.

The methods of the invention can be used to prepare new derivatized compounds useful in various chemical industries. For example, the synthetic method of the present invention can be used to prepare vaccines with multiple antigens attached to a template molecule. In another embodiment of the invention, artificial enzymes can be synthesized by bringing together different peptidic secondary structure elements. The artificial enzyme can contain a catalytic triad in a binding grove. In yet another embodiment of the invention, several saccharides can be attached to a template to form mimics of important oligosaccharides involved in protein transport and cell signaling to be used for regulation of physiological disorders. Furthermore, a molecular template can be derivatised with a variety of pharmacophores to yield multifunctional ligands for complex receptors.

The invention also provides a method of using protection groups to produce microarrays on a solid support. Two or more protection groups are formed comprising building block units linked together. The protection groups are attached to a multiple of distinct locations on a solid support such that at least two of the locations are associated with protection groups having a different number of building block units. The protection groups are attached to the various locations on the solid support through functional groups on the solid substrate. One or more building block unit is removed from each protection group using chemical, electrochemical, or photolytic reactions to form at least one exposed functional group on the solid support. A target group is reacted with the exposed functional group. Additional building block units are consecutively removed from the protection groups remaining. Each exposed functional group on the solid support can be reacted with a different target group. The microarrays can be used for biomolecular screening.

### BRIEF DESCRIPTION OF THE DRAWINGS

The invention can be more completely understood in consideration of the following detailed description of various embodiments of the invention in connection with the accompanying drawings, in which:

FIG. 1 is a drawing of the chemical structure of a protected pentalysine template.

FIG. 2 is a drawing showing the concept of successive UniChemical access to functional groups.

FIG. 3 is an illustration of the linear increase of complexity with number of functional groups using the UCP synthetic scheme compared to the quadratic increase in complexity with number of functional groups using traditional orthogonal protection schemes.

FIG. 4 is a drawing of a N-sec-butyl glycine oligomeric protection group and one type of chemical reaction that can be used to remove the building block units of a protecting group.

FIG. 5 is a drawing of a polyfunctionalized target compound of a pentalysine template formed by removal of the UCP protection groups.

FIG. 6 is a drawing showing the synthesis of a target compound with different peptide sequences attached to a peptide template molecule.

### DETAILED DESCRIPTION OF THE INVENTION

The present invention provides a protected template molecule and a new one-dimensional UniChemo Protection (UCP) organic synthetic method for preparing polyfunctional organic molecules. The synthetic method can be used with many kinds of chemical reactions and provides selective access to many functional groups in a template molecule. The method utilizes protection groups that are each composed of building block units that can be removed one by one affording a new protection group one unit shorter or exposing a functional group on the template molecule. The exposed functional group on the template molecule can react with a target group. Different target groups can be introduced into the template molecule by using protection groups containing different numbers of building block units.

UniChemo Protected Compound

A UniChemo Protected compound (hereinafter “UCP” compound) is the reaction product of a template molecule and various protection groups. The template molecule has more than one functional group. The protection groups are attached to the template molecule through the functional groups of the template molecule. The protection groups comprise building block units linked together and each protection group can contain a different number of building block units. At least two of the protection groups contain different numbers of building blocks. In one embodiment, each protection group contains a different number of repeating block units. The UCP compound can contain up to 1000 protection groups.

The template molecule is a multifunctional compound. Typically, the functional groups are similar. The functional groups can be, for example, a hydroxyl, a thiol, a carboxylate, an amine, an amide, an alkyne, an aldehyde, a ketone, or a mixture thereof. The functional groups can be the same or different. The template molecule can be, for example, a peptide, a glycopeptide, a carbopeptide, a monosaccharide, a oligosaccaride, a DNA fragment, or any organic molecule with more than one functional group. The template can also be a high molecular weight compound such as, for example, a protein, a polysaccharide, or a DNA molecule.

In one embodiment, the template is an oligosaccharide containing 1 to 20 saccharide units. In this embodiment, the protection groups are attached to the template molecule through the functional hydroxyl groups.

In another embodiment, the template molecule is a peptide containing 1 to 20 amino acid units. Suitable amino acids can include, for example, lysine, alanine, glycine, and mixtures thereof. In this embodiment, the protection groups are attached to the template molecule through the functional amine or amide groups. An example of a peptide template is a peptide comprising 5 or more lysines. The template molecule can be a dendrimer; for example, the template can be formed by linking the lysine molecules through amide bonds to both alpha and epsilon nitrogens.

In another embodiment, the template molecule is a DNA fragment. In this embodiment, the protection groups are attached to the template molecule through the functional amine groups of adenine, cytosine, guanine, and thiamine.

In another embodiment, the template is an organic compound such as a substituted steroid, a substituted cubane, a substituted adamantane, a substituted aromatic or heterocyclic compound, substituted alkaloids, and the like. Other suitable organic compounds include polyamines such as spermidine and polyvinyl alcohol.

In yet another embodiment, the template is a solid support. The solid support can be a polymer, copolymer, glass, gold-coated glass, or silica surface. The solid support has functional groups such as, for example, amino, hydroxy, carboxy, or sulfydryl groups. The solid support can be a coating, membrane, plate, particle, bead, and the like. Suitable polymers include, for example, functionalized polyethylene, polypropylene, polystyrene, polycarbonate, polyacrylate, polyurethane, and Teflon™. Specific examples of functionalized polymers include polyethylene and polypropylene with carboxy groups (formed through oxidation with CrO₃), polyethylene and polypropylene with amino groups (formed from carboxy groups), polystyrene with amino groups, polycarbonate with amino groups, and hydroxylated Teflon (formed by threatement with hot concentrated potassium hydroxide). Suitable copolymers include, for example, polyoxypropylene/polyethylene glycol and polyacrylamide/polyethylene glycol. Suitable glasses include controlled pore glass with functional amino groups, gold coated glass with functional sulfydryl groups, and glass treated with amimopropylsilanes.

The protection groups comprise one or more building block units. The building block units are connected together and can be removed consecutively with a chemical, electrochemical, or photolytic reaction. The building blocks are typically linked with a C—X—C bond where X is either NR, O, S, SiR₂, C≡C, O—SiR2-O, PR, O—PO—O, O—PO₂—O, O—CO—O, CONR, NR—CO—O, NR—CO—NR, O—S(O₂), an orthoester, an acetal, a ketal or NR—S(O₂); and R is a hydrogen, an alkyl, an aryl, or an alkoxy group.

The protection group can have one or more building block units but only the terminal building block unit is active for removal at any point in time and the other building blocks are inert. The structure of the active building block unit is typically different from the other building block units. In one embodiment, the active building block unit contains a secondary amine group while the inert building block units contain a substituted amide group. In another embodiment, the active building block unit contains an aniline group while the inert building block units contain an unsubstituted amide group. In another embodiment the active terminal group is a tertiary alcohol while the inert units contain an ester group. In another embodiment the terminal unit is an aldehyde or ketone while the inert units contain an acetal or ketal. In yet another embodiment, the terminal unit contains an ester or an acid while the inert unit contains an orthoester.

In one embodiment, the building block units are amines such as alpha, beta, gamma, or epsilon amino acids. The amino acids are linked together through an amide bond to form protection groups of various lengths. Suitable amino acid protection groups include, for example, oligomers of N-substituted glycine, other N-substituted amino acids, 2-amino-benzoic acid, and (2-amino-phenyl)-acetic acid. The amide group can be substituted with a (C₁ to C₁₀) alkyl or aryl group. Substitution of the amide group can alter the reactivity of the protection group towards removal of one of the building block units and towards the target chemicals used to react with the exposed functional group of the template molecule.

Typical (C₁ to C₁₀) alkyl groups include, for example, methyl, ethyl, isopropyl, sec-butyl, tert-butyl, 1-ethyl-propyl, 1-isopropyl-2-methyl-propyl, adamantyl, isopentyl, and neopently. An alkyl group can be substituted with with halogens or other primary, secondary or tertiary alkyl groups such as methyl, ethyl, isopropyl, sec-butyl sec-pentyl, tert-butyl, benzyl or aryl groups. Examples include oligomers of N-(1-isopropyl-2-methyl-propylamino)acetic acid and N-(1-ethyl-propylamino acid).

Typical aryl groups include phenyl, pyridyl, pyrimidyl, pyroles, pyrolines, imidazoles, triazoles, tetrazoles, thiazoles, oxazoles, pyrazoles, fused aromatic ring-systems based on these entities and the like. The aryl groups can be substituted with either electron donating or electron withdrawing groups such as halo, nitro, cyano, azide, methylsulfinyl, sulfone, methoxy, carboxy and the like.

One class of protection groups is N-alkylated oligoglycine of the formula

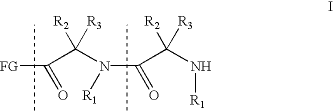

where FG refers to the functional group of the terminal molecule. R₁, R₂, and R₃ are alkyl (e.g. methyl, ethyl, isopropyl, isobutyl, tert-butyl, sec-butyl, neopentyl), aryl (with or without electron withdrawing or electron releasing groups), or heterocyclic groups. The dashed lines show the parts of the protection group that are active and inert. The group to the right of the first dashed line and to the left of the second dashed line is an inert group. The terminal group, the group to the right of the second dashed line, is the active building block unit. Although only one inert building block is shown in the figure, there can be more than one. The number of building block units can range from 1 to 1000. In some embodiments, a protection group can contain only the terminal active building block unit.

Protection groups containing building blocks of N-sec-butylglycine are preferred. However, one additional —CH₂ groups can be inserted to yield N-substituted beta-amino acids. The beta amino acid may similarly have any of the four CH-protons substituted with alkyl, aryl or hetero atom substituents.

Another class of protection groups comprising amino acid building blocks is an oligomer of 2-amino benzoic acid of the formula

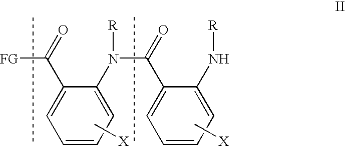

where R is hydrogen or an alkyl substituent (e.g. methyl, ethyl, isopropyl, isobutyl, tert-butyl, sec-butyl, or neopentyl); and X is an electron withdrawing or donating substituent such as methoxy, nitro, or methylsulfinyl. The protection group can contain more than one inert building block unit.

Yet another class of protection groups comprising amino acid building blocks is a (2-amino-phenyl)-acetic acid of the formula

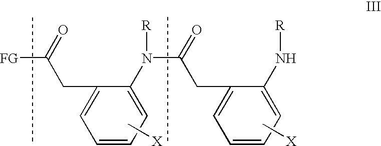

where R is hydrogen or an alkyl substituent (e.g. methyl, ethyl, isopropyl, isobutyl, tert-butyl, sec-butyl, or neopentyl); and X is an electron withdrawing or donating substituent such as methoxy, nitro, or methylsulfinyl. The protection group can contain more than one inert building block unit.

The building block units of the protection groups can be linked with an anhydride group. An example of such a protection group is a compound of formula

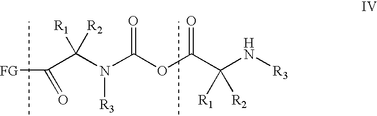

where R₁, R₂, and R₃ are alkyl (e.g. methyl, ethyl, isopropyl, isobutyl, tert-butyl, sec-butyl, neopentyl), aryl (with or without electron withdrawing or electron releasing groups), or heterocyclic groups. The protection group can contain more than one inert building block unit.

The building block units of the protection groups can be linked by a amine group. An example of such a protection group is a compound of formula

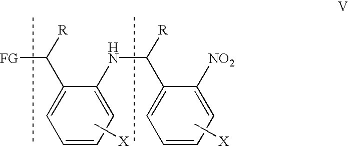

where R is hydrogen or an alkyl substituent (e.g. methyl, ethyl, isopropyl, isobutyl, tert-butyl, sec-butyl, or neopentyl); and X is an electron withdrawing or donating substituent such as methoxy, nitro, or methylsulfinyl. The protection group can contain more than one inert building block unit.

The building block units of the protection group can be linked by urethane. An example of such a protection group is of the formula

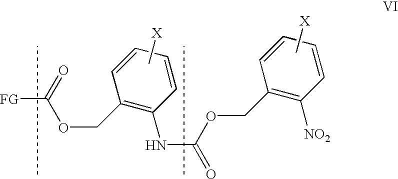

where X is an electron withdrawing or donating substituent such as methoxy, nitro, or methylsulfinyl. The protection group can contain more than one inert building block unit.

Other protection groups can have the structure

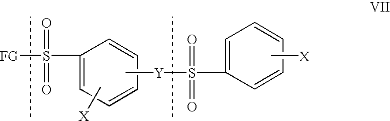

where X is an electron withdrawing or donating substituent such as methoxy, halogen, nitro, or methylsulfinyl and Y is NH, O, O—CH₂—CH₂. The protection group can contain more than one inert building block unit.

The building block units of the protection group can be connected through silane groups. A protection group containing silane groups is of the formula

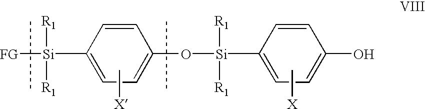

where R₁ is hydrogen or an alkyl substituent (e.g. methyl, ethyl, isopropyl, isobutyl, tert-butyl, sec-butyl, or neopentyl); and X is an electron withdrawing or donating substituent such as methoxy, nitro, or methylsulfinyl. The protection group can contain more than one inert building block unit.

Another structure for a protection group is of the formula

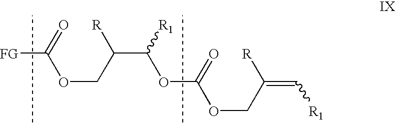

where R and R₁ are from the group of alkyl (e.g. methyl, ethyl, isopropyl, isobutyl, tert-butyl, sec-butyl, neopentyl), aryl (with or without electron withdrawing or electron releasing groups), or heterocyclic moieties. The protection group can contain more than one inert building block unit.

An ester bond can connect the building block units of the protection as shown in formula

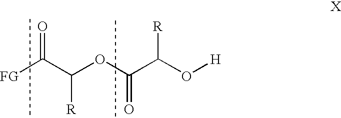

and formula

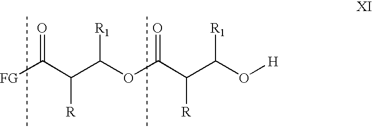

where R and R₁ are alkyl (e.g. methyl, ethyl, isopropyl, isobutyl, tert-butyl, sec-butyl, neopentyl), aryl (with or without electron withdrawing or electron releasing groups), or heterocyclic moieties. The protection group can contain more than one inert building block unit. The functional group of the template molecule could be a sterically more hindered alcohol or a aromatic hydroxyl group. The building block units can be removed by conversion of the terminal hydroxyl group to a group that may be removed with thiourea or the CS₂/hydrazine adduct.

Another type of protection group building block units are saccharides. Any saccharide can be used including, for example, glucose, fructose, sucrose, maltose, and the like.

One scheme for introducing different length oligomers onto the template molecule to form a UCP compound involves adding a different length protection group to the various monomers that form the template molecule followed by reaction of the monomers to form the template molecule. For example, a UCP compound containing a pentalysine template molecule with five different length protection groups of N-sec-butyl-glycine can be synthesized by reacting the epsilon nitrogen of lysine with N-sec-butyl-glycine. The building block unit of N-sec-butyl-glycine can be synthesized by reacting ethyl bromoacetate and sec-butyl amine followed by ester hydrolysis. The secondary amine of N-sec-butyl-glycine can be protected by reaction with a solution of 9-fluorenylmethyloxycarbonyl chloride (Fmoc-Cl) to produce N-fluoren-9-ylmethoxy N-sec-butyl glycine. The carboxylic group on the glycine then can react with the epsilon nitrogen of lysine.

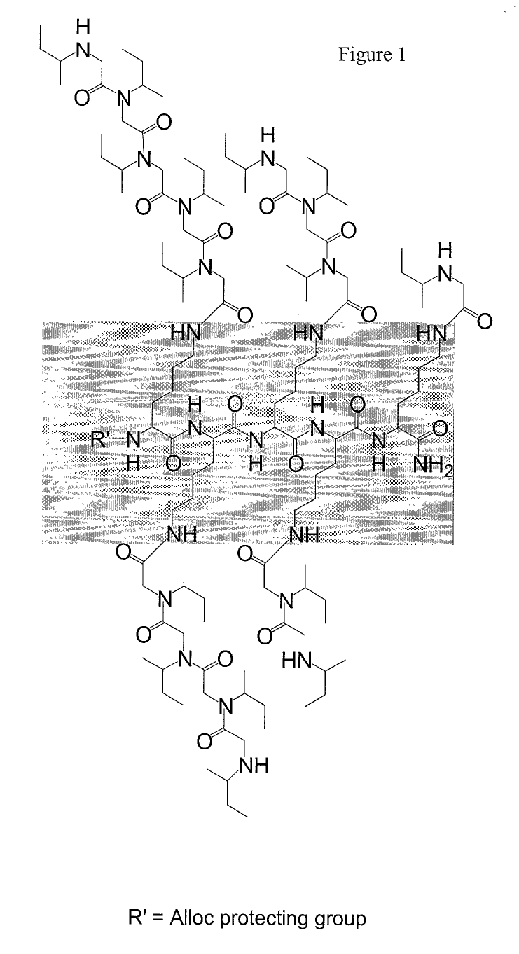

To ensure quantitative and selective placement of the protection group on the epsilon nitrogen of lysine, a blocking group can be placed on the alpha nitrogen of lysine. Typical blocking groups include 9-fluorenylmethyloxycarbonyl (Fmoc) and allyl carbonate (Alloc). Further, the lysine can be attached to a resin through the carboxylic acid group. After addition of one building block unit of N-sec-butyl-glycine to the epsilon nitrogen of lysine, one-fifth of the product is removed. Then the remaining material is reacted with enough N-sec-butyl-glycine to attach another building block unit. After removing another one-fifth of the product containing two building block units, another building block unit of N-sec-butyl-glycine is added. This process is continued until one-fifth of the material has one building block unit, one-fifth of the material has two building block units, one-fifth of the material has three building block units, one-fifth of the material has four building block units, and one-fifth of the material has five building block units of the protection group. The lysine compounds containing various numbers of building block units of the protection group are then removed from the resin and reacted with each other after the blocking group on the alpha nitrogen of the lysine is removed. A UCP compound is formed comprising five different length protection groups attached to the epsilon nitrogens in the pentalysine template. This synthetic process is described further in Examples 1 to 4. FIG. 1 is a drawing of the chemical structure of the protected pentalysine template. Resins applicable to peptide, organic, and oligosaccaride chemical synthesis can also be used with UCP strategies. Suitable resins include, but are not limited to, TentaGel™ (available from Peptides International, Inc., Louisville, Ky.), Argogel™ (available from Aronault Technologies, Inc., San Carlos, Calif.), REM (benzyloxyacrylate resins), polyethylene glycol (PEG)-based resins, polyethylene glycol/polyamide (PEGA)-based resins, and Wang resins (4-benzyloxybenzyl alcohol resins).

A similar synthetic method can be followed using different amino acids or a mixture of amino acids to form the template molecule. Likewise, a similar synthetic scheme can be used substituting a saccharide in place of lysine as the template monomer. With saccharides, the different length protection groups can be added to a single monosaccharide or a mixture of saccharides. After the protection groups have been attached, the saccharides can be reacted to form an UCP compound comprising an oligosaccharide template with various lengths of protection groups attached to the hydroxyl groups of the oligosaccharide.

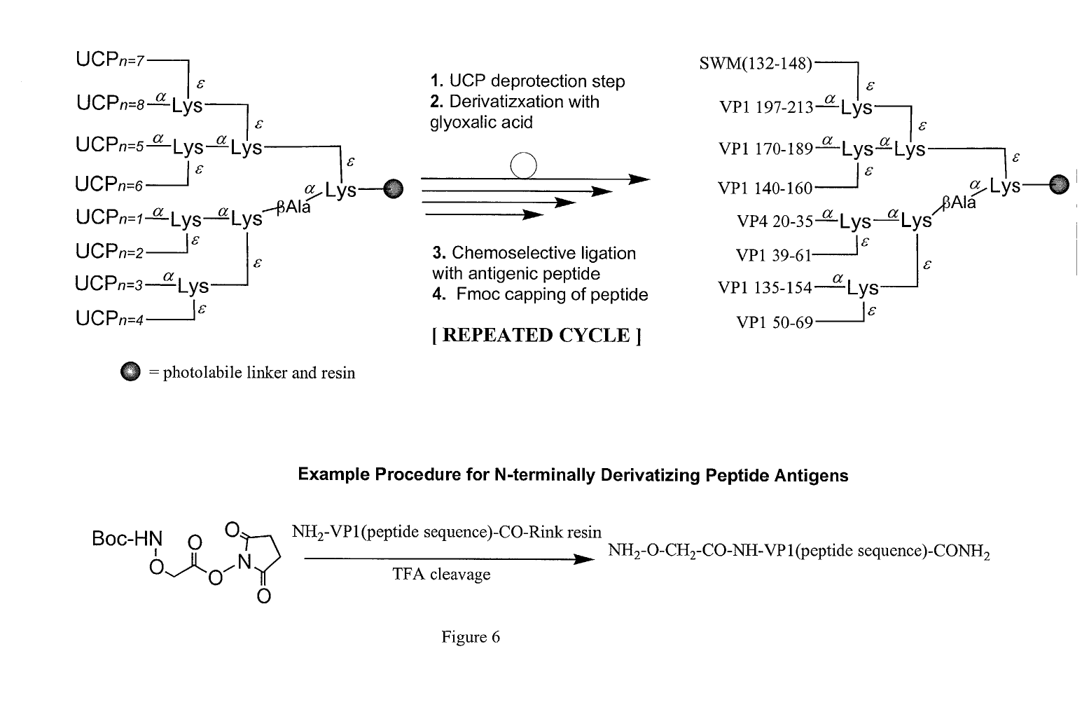

Any monomer with several functional groups could be reacted with various lengths of protection groups and then reacted to form an oligomer with many different length protection groups. The structure of the template itself can be complex. FIG. 6 shows a template comprising one alanine molecule and seven lysine molecules. The lysine molecules can be linked to each other through an amide bond with either the alpha or epsilon nitrogen resulting in the formation of a branched or dendrimer template.

Method of Preparing Target Compound with Different Functionality

The invention also provides methods of preparing a target compound with different functionality. In one embodiment, a target compound with different functionality can be formed by initially forming protection groups comprising building block units that are linked together. A protected compound is formed containing two or more protective groups having a different number of building block units. A terminal building block unit is removed from each protection group using one or more chemical, electrochemical, or photolytic reactions. Additional building blocks are consecutively removed from each building block unit. As each protection group is completely removed, the newly formed intermediate compound can react with a target group.

Another embodiment involves initially forming a UCP compound that is the reaction product of a template molecule having more than one functional group and various protection groups. The protection groups are attached to the template molecule through the functional groups of the template molecule. The protection groups comprise building block units linked together and each protection group can contain a different number of building block units. The protection group has at least one building block unit. The terminal building block unit is active and the remaining units are inert.

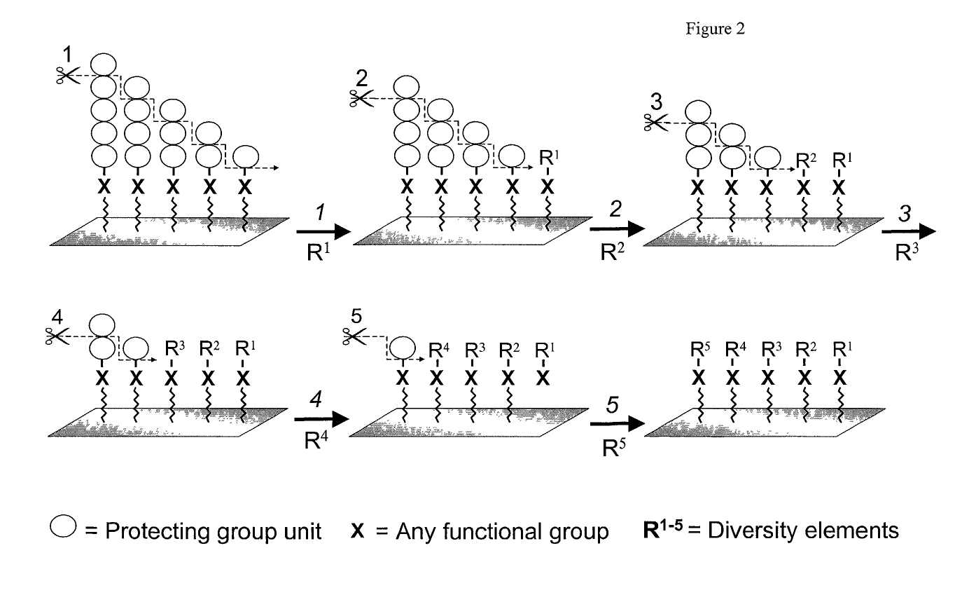

Chemical, electrochemical, or photolytic reactions are used to remove the active building block unit from each protection group as shown in FIG. 2. For protection groups containing at least two building block units, the removal of the active group results in the formation of a shorter protection group by one building block unit. Alternatively, the removal of the active group from a protection group containing only one building block unit results in the complete removal of the protection group from a functional group of the template molecule. The removal of a protection group from a functional group of the template molecule results in an exposed functional group of the template molecule that can react with a desired target group. Only functional groups of the template unit without a protection group can react with the desired target group. The desired target group and the protection groups are chosen such that the target group does not react with the protection group.

Additional building block units are consecutively removed from the protection groups using chemical, electrochemical, or photolytic reactions to form even shorter protection groups and at least one additional exposed functional group of the template molecule that is not attached to a protection group. The newly exposed functional group is then reacted with another desired target group. The second target group added is generally different than the first target group. The process of removing one building block unit of the protection group can be repeated and followed by the reaction of any exposed functional group of the template molecule with another target group.

The functional group of the template compound and the active building block unit on the protection group typically have a large difference in reactivity. This difference is usually either steric or electronic. For example, one group can be more electrophilic or nucleophilic than the other group. Examples of groups with different reactivity include 1) a sterically crowded secondary amine and a primary amine; 2) an amine and a nitro; 3) an aromatic and an aliphatic amine; 4) a ketone and an aldehyde; 5) an alcohol and a carboxylic acid; 6) a carbamate or a urethane and a carbonate; 7) a secondary or tertiary alkene and a primary alkene; and 8) an alkyne and an alkene.

After one building block unit has been removed from each of the protection groups remaining attached to the template molecule, there is a new active group on the protection group and one fewer inert building block unit. In some embodiments, the active group is chemically altered to make it less reactive with the target compounds that are reacted with functional groups of the template compound. For example, an active building block unit containing an amine group can be oxidized to a nitro group.

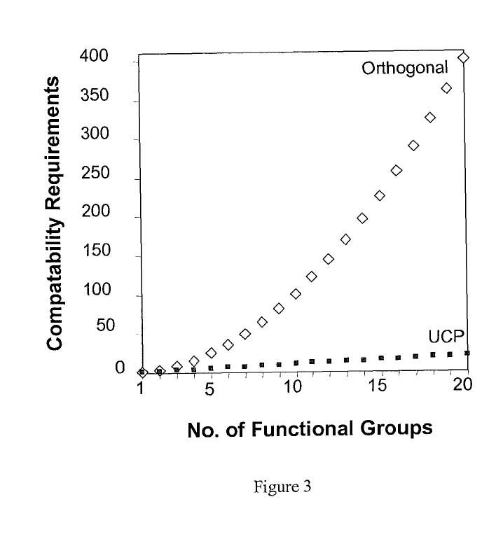

The UniChemo Protection scheme is much simpler than that typically associated with orthogonal protection as shown in FIG. 3. Because the UCP synthetic scheme is fundamentally based on uniform reactions to remove the protection groups, the requirement of reaction compatibility with other parts of a molecule increases linearly with the number of protected functional groups. In contrast, compatibility requirements with other parts of a molecule increase in a quadratic manner with the number of protected functional groups using traditional orthogonal protection schemes. The UniChemo Protection scheme offers distinct advantages when the number of functional groups exceeds about five. The UCP synthetic scheme provides an orthogonal protection process that is not dependent on a multitude of different orthogonal chemistries.

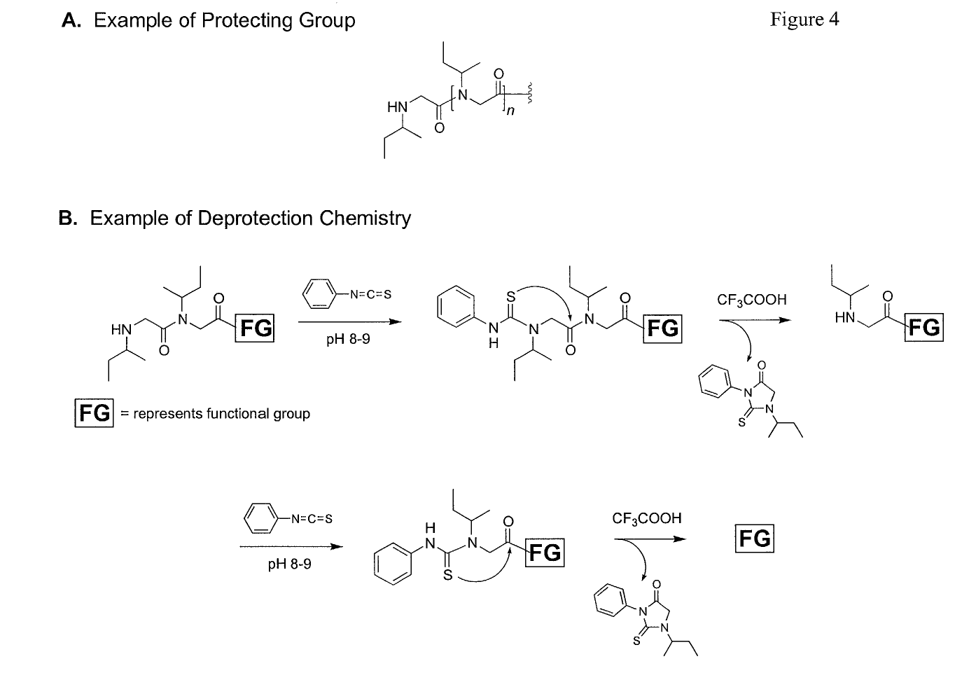

In one embodiment shown in FIG. 4, oligomers of N-isobutyl glycine can be used as the protection group. The active building block unit is removed by a reaction with phenyl isothiocyanate using the well-characterized Edman degradation reaction. Under basic pH conditions, phenyl isothiocyanate reacts with the secondary amine of the active building block unit. The addition of an acid such as trifluoroacetic acid (TFA) results in the formation of a five-membered ring and the cleavage of the active group from the protection group. The process shortens the protection group by one building block unit and produces a new active group.

The UCP compound can be used to form a target compound containing multiple antigen peptides (MAP). As each protection group is removed, a peptide chain can react with the exposed functional group of the template molecule. Different peptide chains can be placed on each functional group of the template molecule. For example, the target groups can be multiple antigens. MAP molecules can be used as vaccines. All possible T-cell determinants and B-cell determinants from a bacterial or viral target protein can be assembled on the same template dendrimer. Furthermore, a sequence that targets specific importer molecules on macrophages, T-cells or B-cells may be assembled on the same MAP. The dendrimer or template molecule is not limited to lysine containing compounds.

Functional synthetic de novo proteins can be prepared using the UCP synthetic scheme. De novo proteins are synthetic proteins that do not occur in nature. Such proteins can be prepared using a protected template such as, for example, a cyclic peptide with four lysine residues. Functional secondary structure elements such as, for example, α-helices or β-sheets can be attached to form folded structures e. g. four helix bundles. One particular useful application of synthetic de novo proteins are enzymes where a catalytic triad (e.g. Ser, His, Asp) is included into a region of the protein able to bind putative substrates. These proteins are synthesized with great difficulty and often with duplication of some of the functional chains with traditional synthetic approaches due to lack of orthogonality during assembly.

In yet another embodiment of the invention, several saccharides can be attached to a template to form mimics of important oligosaccharides involved in protein transport and cell signaling to be used for regulation of physiological disorders.

The invention also provides a method of using protection groups to produce microarrays on a solid support. Two or more protection groups are formed comprising building block units linked together. The protection groups are attached to a multiple of distinct locations on a solid support such that at least two of the locations are associated with a protection group having a different number of building block units. The protection groups are attached to the various locations on the solid support through functional groups on the solid substrate. One building block unit is removed from each protection group using chemical, electrochemical, or photolytic reactions to form at least one exposed functional group on the solid support. A target group is reacted with the exposed functional group. Additional building block units can be consecutively removed from the protection groups remaining. Each exposed functional group on the solid support can react with a different target group. The microarrays can be used for biomolecular screening.

Various arrays can be attached to the exposed functional group of the solid support. Such microarrays can be used for biomolecular screening. For example, DNA and LNA arrays can be attached and used with various hybridization and PCR techniques. DNA, LNA, and PNA arrays can be attached and used for diagnosis. Protein arrays can be attached and used for parallel ligand screening. Antibody arrays can be attached and used for immunoassays. Peptide arrays can be attached and used for screening and diagnosis. Oligosaccharide arrays can be attached and used for lectin screening.

Completely unnatural libraries of scaffolds such as calixarenes can also be derivatized with different protection groups on each functional group and a variety of recognition motifs can be incorporated on the templates. Furthermore, a molecular template can be derivatized with a variety of pharmacophores to yield multifunctional ligands for complex receptors. This approach will induce asymmetry into host guest interactions, a field of scientific and commercial opportunity.

The synthetic methods of the invention can be automated and used for combinatorial synthesis of complex molecules. Compared to conventional synthesis this is a considerable advantage. The linear assembly of template molecules to form UCP compounds as well as the further manipulation and derivatization with various target groups can be automated. An advantage of the UCP synthetic scheme is the common set of chemical reactions used for removal of all the protection groups.

With respect to the above description, it is to be realized that the optimum dimensional relationships for the molecular entities of the invention, to include variations in size, structure reactivity, function and manner of operation, assembly and use, are deemed readily apparent and obvious to one skilled in the art, and all equivalent relationships to those illustrated in the drawings and described in the specification are intended to be encompassed by the present invention.

### EXAMPLES

### Example 1

### Preparation of sec-butylamino-acetic Acid Ethyl Ester

Synthesis was carried out in analogous manner to the literature procedure: J. A Kruijtzer, L. J. F. Hofmeyer, W. Heerma, C. Versluis, R. M. J. Liskamp, Chem. Eur. J., 4(8), pp. 1570-1580 (1998). Ethyl bromoacetate (50 mmol) in tetrahydrofuran (25 mL, THF) was added dropwise to a cooled solution of sec-butylamine (110 mmol) in THF (25 mL) over 5 min. After stirring for 4 h at room temperature, the reaction mixture was concentrated in vacuo and resuspended in dry diethyl ether. The mixture was filtered to remove sec-butylamine hydrobromide, the residue washed with ether, and the filtrate concentrated in vacuo. Yield 91% as a colourless oil. ¹H NMR (250 MHz, 298° K, CDCl₃, ppm) δ0.87 (t, 3H, J=7.5 Hz), 1.00 (d, 3H, J=6.3 Hz), 1.25 (t, 3H, J=7.2 Hz), 1.43 (m, 2H), 1.83 (br, 1H), 2.52 (m, 1H), 3.38 (s, 2H), 4.16 (q, 2H, J=7.2 Hz); ¹³C NMR (62.90 MHz, 298° K, CDCl₃) 10.24, 14.32, 19.66, 29.58, 54.32, 60.81, 172.86.

### Example 2

### Synthesis of N-fluoren-9-ylmethoxycarbonyl-N-sec-butyl-glycine

Sodium hydroxide (NaOH, 4N, 2.50 mL) was added to a solution of sec-butylamino-acetic acid ethyl ester (Example 1, 10 mmol) in dioxane (35 mL) and methanol (12.5 mL). After stirring for 30 min at room temperature the reaction mixture was concentrated in vacuo. The sodium salt was dissolved in water and the pH adjusted to 9-9.5 with concentrated hydrochloric acid. To this mixture a solution of 9-fluorenylmethyloxycarbonyl chloride (Fmoc-Cl, 10 mmol) in 1,2-dimethoxyethane was added in one portion. Stirring was continued for 3 h, and the pH was maintained between 8.5-9.5 by the addition of triethylamine. The reaction mixture was concentrated in vacuo to remove 1,2-dimethoxyethane , and the residue poured was poured onto 20% (w/v) citric acid (120 mL). The aqueous layer was extracted with ethyl acetate (4×60 mL) and the combined organic layers were washed with water, brine, dried (MgSO₄), and concentrated in vacuo. Column chromatography (silica, eluent: DCM:MeOH (9:1) and DCM:MeOH:AcOH (90:9.5:0.5) gave as a white solid. Yield 71%. R_{f} 0.56 (eluent DCM:MeOH:AcOH (90:9.5:0.5)). The NMR spectra clearly show the presence of both rotamers. ¹H NMR (250 MHz, 298° K, CDCl₃, ppm) d 0.83, 0.73 (t, 3H, J=7.2 Hz), 1.00, 1.06 (d, 3H, J=6.9 Hz), 1.14-1.49 (m, 2H), 3.60-3.99, 4.09-4.26 (m, 4H), 4.35-4.58 (m, 2H), 7.22-7.40 (m, 4 Har), 7.48-7.59 (m, 2Har), 7.64-7.76 (m, 2Har), 10.39 (b, 1H); ¹³C NMR (62.90 MHz, 298° K, CDCl₃) d 10.93, 18.03, 18.32, 27.48, 27.73, 43.28, 43.99, 47.30, 53.35, 67.58, 119.23, 124.89, 127.05, 127.62, 127.67, 141.29, 141.39, 143.97, 156.74, 156.06, 175.11, 175.33. Calc. for C₂₁H₂₃NO₄ 353.16 (monoisotopic), Exp. ESMS: 353.2 Da.

### Example 3

### Synthesis of the Alloc-Lysine(Fmoc-UCPₙ₌₁₋₅)-OH Building Blocks

The UCP-unit building block can be used in solid phase assembly of UCP-protected amino acids for solid phase assembly of UCP-protected templates. Using the product from Example 2, mono to pentamer protected lysines were synthesized on solid support. High yields of oligomeric N-sec-butylglycyl protection groups are readily obtained using strong activation during amide-bond formation on the solid-support. Solid-phase peptide chemistry and solid-phase organic chemistry were performed in flat-bottom luer syringes fitted with sintered Teflon filters (50 μm pore size). All solvents were purchased from Labscan Ltd. (Dublin, Ireland) stored over 3 Å molecular sieves.

Wang resin (1.01 g, loading: 0.83 mmol/g) was swollen in N,N-dimethylformamide (DMF) for 10 min, and washed with 20% N,N-diisopropylethylamine (DIPEA) in DMF and dry dichloromethane (DCM). The resin was lyophilized for 4 h and then acylated twice with N^{α}-Fmoc-Lys(N^{ε}-Alloc)-OH (2 mmol), 1-(mesitylene-2-sulfonyl)-3-nitro-1H-1,2,4-triazole (MSNT, 2 mmol) and N-methylimidazole (MeIm, 4 mmol) in dry DCM (10 mL). The resin was then thoroughly washed (typically 15 reaction vessel volumes) with DCM and DMF.

For Fmoc removal the resin was treated twice (2×10 min) with 20% piperidine in DMF. After thorough washing with DMF, the newly liberated alpha-amine was protected by 4 treatments with trityl chloride (2 mmol) and DIPEA (4 mmol) in DCM 10 mL.

After washing with DCM and lyophilization for 16 h, the N^{ε}-Alloc group was removed with two treatments of tetrakis(triphenylphosphine)palladium(0) ((PPh₃)₄Pd, 405 mmol), N-ethylmorpholine (NEM)-acetic acid (9.5:10) in DCM for 1 h at room temperature under argon atmosphere.

After washing the resin with DCM and DMF, the N^{ε}-amino group was acylated with N-fluoren-9-ylmethoxycarbonyl-N-sec-butyl-glycine (0.913 mmol, 1.1 equiv) using N,N,N′,N′-tetramethyl-O-(benzotriazol-1-yl)uronium tetrafluoroborate (TBTU) (0.910 mmol)/NEM (2.7 mmol) in DMF for 14 h under an argon atmosphere. The temporary N^{α}-trityl group was removed by 3 treatments with 0.1% TFA in DCM for 20 min. Following a DCM wash, the resin was treated twice with a solution of allyl chlorofornate (2.8 mmol) and NEM (4.7 mmol) in cold anhydrous DCM (8 mL) for 1 h at room temperature.

A portion of the growing N^{α}-Alloc-Lys(N^{ε}-Fmoc-UCP)ₙ-OH oligomer is cleaved off from an aliquot of the resin after each addition of a N-sec-butylglycine unit, and the building block is used to assemble a multifunctional UCP-protected template on solid support. After washing with DCM, approximately 20% of the resin was separated and cleaved from the resin with 98% TFA for 1.5 h at room temperature to give N^{α}-Alloc-Lys(N^{ε}-(N-fluoren-9-ylmethoxycarbonyl-N-sec-butyl-glycine))-OH after reversed phase high-performance liquid chromatography (RP-HPLC). Chromatographic separations were achieved using linear gradients of buffer B in A (A=0.1% aqueous TFA; B=90% CH₃CN, 10% H₂O, 0.09% TFA), 0-60% over 80 min at a flow rate of 10 mL/min (preparative) on a Waters 600E solvent delivery system equipped with a (C₁₈, 2.5×25 cm, Millipore Delta Pak 15 μm) column, and a Waters M-991 photodiode array detector.

The remaining 80% of the resin was used for the preparation of higher oligomerized protecting groups (on the side-chain of the lysine core). For the N^{ε}-lysine-derivatized dimeric protected group, the Fmoc group was deprotected with 20% piperidine in DMF for 20 min and following DMF washing N-fluoren-9-ylmethoxycarbonyl-N-sec-butyl-glycine (0.900 mmol) was coupled with bromo-trispyrrolidinophosphonium hexafluorophosphate (PyBroP, 0.899 mmol) and DIPEA (1.8 mmol). After washing with DCM, approximately 20% of the resin was separated and the product cleaved from the resin with 98% (v/v) TFA for 1.5 h at room temperature to give N^{α}-Alloc-Lys(N^{ε}-(di[N-fluoren-9-ylmethoxycarbonyl-N-sec-butyl-glycine]))-OH after HPLC purification as described above.

For the N^{ε}-lysine-derivatized trimeric protected group, the Fmoc group was deprotected with 20% piperidine in DMF for 20 min and following DMF washing N-fluoren-9-ylmethoxycarbonyl-N-sec-butyl-glycine (0.675 mmol) was coupled with bromo-tris-pyrrolidino-phosphonium hexafluorophosphate (PyBroP, 0.670 mmol) and DIPEA (1.4 mmol). After washing with DCM, approximately 20% of the resin was separated and the product cleaved from the resin with 98% TFA for 1.5 h at room temperature to give N^{α}-Alloc-Lys(N^{ε}-(tri[N-fluoren-9-ylmethoxycarbonyl-N-sec-butyl-glycine]))-OH after HPLC purification as described above.

For the lysine-derivatized tetrameric protected group, the Fmoc group was deprotected with 20% piperidine in DMF for 20 min and following DMF washing N-fluoren-9-ylmethoxycarbonyl-N-sec-butyl-glycine (0.450 mmol) was coupled with bromo-tris-pyrrolidino-phosphonium hexafluorophosphate (PyBroP, 0.445 mmol) and DIPEA (0.9 mmol). After washing with DCM, approximately 20% of the resin was separated and the product cleaved from the resin with 98% TFA for 1.5 h at room temperature to give N^{α}-Alloc-Lys(N^{ε}-(tetra[N-fluoren-9-ylmethoxycarbonyl-N-sec-butyl-glycine]))-OH after HPLC purification as described above. For the lysine-derivatized tetrameric protected group, the Fmoc group was deprotected with 20% (v/v) piperidine in DMF for 20 min and following DMF washing N-fluoren-9-ylmethoxycarbonyl-N-sec-butyl-glycine (0.225 mmol) was coupled with bromo-tris-pyrrolidino-phosphonium hexafluorophosphate (PyBroP, 0.225 mmol) and DIPEA (0.5 mmol).

After washing with DCM, approximately 20% of the resin was separated and the product cleaved from the resin with 98% (v/v) TFA for 1.5 h at room temperature to give N^{α}-Alloc-Lys(N^{ε}-(penta[N-fluoren-9-ylmethoxycarbonyl-N-sec-butyl-glycine]))-OH after HPLC purification as described above.

The UCP-protected lysine building blocks can be used in assembly of templates in which there is independent access to a number of functional groups. The synthesis is performed on solid phase and all reactions involved are essentially quantitative as measured by HPLC and MS on the product after cleavage off the resin. Molecular dynamics simulations indicate that low energy conformers of N-sec-butylglycyl protecting group oligomers are generally flexible, extended, and hydrophobic, and agrees well with experimental observations in terms of accessibility and solubility in organic solvents.

### Example 4

### Synthesis of Unichemo Protected Compound

For the attachment of the pentalysine template to the solid-support a photolabile 4-4-[1-(9H-fluoren-9-ylmethoxycarbonylamino)-ethyl]-2-methoxy-5-nitro-phenoxy-butanoic aminomethy linker was used. N^{α}-Alloc-Lys(N^{ε}-(N-fluoren-9-ylmethoxycarbonyl-N-sec-butyl-glycine))-OH (10.4 μmol) was coupled to 4-4-[1-(9H-fluoren-9-ylmethoxycarbonylamino)-ethyl]-2-methoxy-5-nitro-phenoxy-butanoic aminomethyl polystyrene resin (1.4 mmol/g; photolabile linker) with O-(7-azabenzotriazol-1-yl)-N,N,N′,N′-tetramethyluronium hexafluorophosphate (HATU, 10.4 μmol) and N,N-diisopropylethylamine (DIEA, 41.6 μmol) in DMF at a concentration of 0.3 M at room temperature for 3 h. After washing with DMF and DCM and lyophilization for 16 h, the N^{ε}-Alloc group was removed with two treatments of tetrakis(triphenylphosphine)palladium(0) ((PPh₃)₄Pd, 0.2 equiv 2.08 μmol), phenylsilane (250 μmol) in DCM for 20 min at room temperature under an argon atmosphere.

After DCM and DMF washes, N^{α}-Alloc-Lys(N^{ε}-(di[N-fluoren-9-ylmethoxycarbonyl-N-sec-butyl-glycine]))-OH (10.6 μmol) was coupled to with HATU 10.6 μmol) and N,N-diisopropylethylamine (DIEA, 40.2 μmol) in DMF at a concentration of 0.3 M at room temperature for 4 h. After washing with DMF and DCM and lyophilization for 20 h, the N^{ε}-Alloc group was removed with two treatments of tetrakis(triphenylphosphine)palladium(0) ((PPh₃)₄Pd, 0.2 equiv 2.08 μmol), phenylsilane (250 μmol) in DCM for 30 min at room temperature under an argon atmosphere.

After DCM and DMF washes, N^{α}-Alloc-Lys(N^{ε}-(tri[N-fluoren-9-ylmethoxycarbonyl-N-sec-butyl-glycine]))-OH (10.6 μmol) was coupled to with HATU 10.4 μmol) and DIPEA (40.2 μmol) in DMF at a concentration of 0.3 M at room temperature for 15 h. After washing with DMF and DCM and lyophilization for 20 h, the N^{ε}-Alloc group was removed with two treatments of tetrakis(triphenylphosphine)palladium(0) ((PPh₃)₄Pd, 0.2 equiv 2.08 μmol), phenylsilane (250 μmol) in DCM for 45 min at room temperature under an argon atmosphere.

After DCM, THF, THF/H₂O (9:1), THF and DMF washes, N^{α}-Alloc-Lys(N^{ε}-(tetra[N-fluoren-9-ylmethoxycarbonyl-N-sec-butyl-glycine]))-OH (10.4 μmol) was coupled to with HATU 10.6 μmol) and DIEA (46.0 μmol) in DMF at a concentration of 0.3 M at room temperature for 5 h. After washing with DMF and DCM and lyophilization for 20 h, the N^{ε}-Alloc group was removed with two treatments of tetrakis(triphenylphosphine)palladium(0) ((PPh₃)₄Pd, 0.2 equiv 2.08 μmol), phenylsilane (250 μmol) in DCM for 30 min at room temperature under an argon atmosphere.

After DCM and DMF washes, N^{α}-Alloc-Lys(N^{ε}-(penta[N-fluoren-9-ylmethoxycarbonyl-N-sec-butyl-glycine]))-OH (10.4 μmol) was coupled to with HATU 10.6 μmol) and DIEA (46.0 μmol) in DMF at a concentration of 0.3 M at room temperature for 5 h. Global Fmoc deprotection was carried out with 50% (v/v) piperidine in DMF to liberate the secondary amines of the protecting groups after scaffold assembly to yield the serially UCP derivatized pentalysine scaffold. An analytical sample was cleaved from the solid-support with 98% (v/v) TFA for 1 h at room temperature. Following evaporation of the TFA, resuspension in 70% (v/v) acetonitrile/H₂O and filtration the sample was characterized by mass spectrometry. Calc. for C₁₂₅H₂₃₃N₂₅O₂₂ 2437.78 (monoisotopic), Exp. ESMS: 2437.8 Da. MALDI-TOF: 2437.7 Da.

The assembly may take place on a solid support allowing easy manipulation and product retrival between synthetic steps. However, in another embodiment of the invention such assembly can be carried out in solution.

### Example 5

### Synthesis of Polyfunctionalized Product

For UCP deprotection cycles, efficient step-wise removal of terminal protecting group units is facilitated by a simple and reliable two-step procedure originally developed by Edman for protein sequencing (Edman, P. Acta Chem.Scand. 10, 761. (1956)). In the first step, phenylisothiocyanate (PITC) reacts quantitatively at pH 8 with the terminal unit of the oligomeric protecting group. In the second step, a quantitative cyclization and elimination reaction occurs at acidic pH, to give the shortened protecting group via the expulsion of a phenylthiohydantoin derivative (FIG. 4).

The effectiveness of the UniChemo Protection strategy was illustrated by the derivatization of a pentalysine-based amino functionalized scaffold on the solid-support. With conventional protection strategies, the controlled derivatization of five or more amino groups on the solid-support is very difficult. This problem was solved by using UCP in the form of N^{ε}-oligo(N-sec-butylglycyl) protected lysine template units for the assembly of the UCP compound (FIG. 1). Following assembly of the UCP compound, all five primary amino groups on the scaffold were successively liberated with PITC/TFA deprotection cycles.

First of all, the UCP compound was subjected to three treatments of 25% (v/v) phenylisothiocyanate (PITC), 10% (v/v) N-methylpiperidine (NMP) in DMF for 30 min at 55° C. After each deprotection reaction, the resin was washed with DMF then DCM and treated twice with excess neat TFA at 30° C. for 30 min. The efficiency of the deprotection step is >98% as determined by reversed-phase HPLC for the truncation of a model trimeric protecting group.

Each newly liberated amino group was acylated with a different carboxylic acids in the following order: β-naphthoic acid, thymine-1-acetic acid, thiophene-2-carboxylic acid, shikimic acid, and Boc-(L)-1,2,3,4-tetrahydroisoquinoline-3-carboxylic acid. The acids (1.05 equiv) were coupled with 2-(1H-benzotriazole-1-yl)-1,1,3,3-tetramethyluronium tetrafluoroborate (TBTU; 1.05 equiv, 0.05 M), DIEA (4 equiv), and DMF for 25 min at room temperature. After coupling the resin was washed with DMF and the PITC-mediated deprotected cycle repeated until all group were deprotected.

Before cleavage from the resin, the N^{α}-Alloc group was removed. After washing with DMF and DCM and lyophilization for 6 h, the N^{ε}-Alloc group was removed with two treatments of tetrakis(triphenylphosphine)palladium(0) ((PPh₃)₄Pd, 0.2 equiv 2.08 μmol), phenylsilane (250 μmol) in DCM for 30 min at room temperature under an argon atmosphere. The target compound was cleaved from the solid-support in methanol with UV irradiation for 3 hours, extracted with 70% (v/v) acetonitrile in water, filtered and then purified by reversed phase high-performance liquid chromatography (RP-HPLC). RP-HPLC was performed on a Waters 110 solvent delivery system equipped with a Schimadzu UV absorbance or a Waters M-991 photodiode array detector and recorded on a PC computer using TurboChrom Navigator 4.1 (Perkin Elmer). Analytical RP-HPLC was performed on Zorbax C₁₈ (5 μm, 0.46 cm×5 cm) column. Chromatographic separations were achieved using linear gradients of 0-80% buffer B in A (A=0.1% aqueous TFA; B=90% CH₃CN, 10% H₂O, 0.09% TFA) over 40 min at a flow rate of 1 mL/min. After the five derivatization steps, cleavage from the solid-support afforded the desired molecule in good purity and yield after reversed phase high-performance liquid chromatography (RP-HPLC).

Characterization of product: ¹H NMR (500.09 MHz, 298° K, CD₃OD, ppm) Due to spectral degeneracy and overlap, characteristic resonances of the side chain groups are reported (FIG. 9): Thymine moiety: 1.84, 4.37, 7.36; Shikimic moiety: 2.15, 2.71, 3.66, 4.35, 6.38; Thiophene moiety: 7.07, 7.59, 7.64; Naphthyl moiety: 7.85, 7.92, 7.95, 8.35; Tetrahydroisoquinoline moiety: 4.54, 7.14, 7.22. Calc. for C₇₀H₉₄N₁₄O₁₅S, 1402.67 (monoisotopic). Exp. ESMS: 1402.7 Da. MALDI-TOF: 1402.6 Da. HPLC crude purity estimate 62%. Isolated yield 26%, from starting resin loading value and after reversed phase-HPLC purification.

Electrospray mass spectra were acquired on a Hewlett-Packard HP1100-MSD mass spectrometer equipped with an atmospheric pressure ionization source. Samples dissolved in 50% aqueous acetonitrile (3 μL) were injected into a moving solvent (100 μL/min; 50:50 0.3% acetic acid in water/0.03% acetic acid in acetonitrile) coupled directly to the ionization source via a fused silica capillary interface (50 μm i.d.×25 cm length). Sample droplets were ionized at a positive potential of 5 kv and entered the analyzer through an interface plate and subsequently through an orifice (100-120 μm diameter) with a capillary potential of 90 V. Full scan mass spectra were acquired over the mass range of 150-1000 Da with a scan step size of 0.1 Da. Molecular masses were derived from the observed m/z values using the HP LC/MSD Chemstation Rev A.06.03 software packages (HP, USA).

Matrix-assisted laser desorption ionization time of flight (MALDI-TOF) mass spectrometry were acquired on a Bruker Reflex™ III MALDI-TOF mass spectrometer. Spectra were obtained (1-100 pulses) using the lowest power required to facilitate desorption and ionization. Ions were accelerated toward the discrete dynode multiplier detector with an acceleration voltage of 20 kV. The matrix -cyano-4-hydroxycinnamic acid (CHC, 10 mg in 1 mL of 70% acetonitrile) was used. Bradykin (1060.2 Da), renin (1759.0 Da), and mellitin (2846.5 Da) were used as the standards for internal calibration of the mass spectra. Beads were irradiated on stainless steel targets with a strong UV lamp for 60 min. The analyte was extracted on the target from the beads using 0.5 μL of 70% acetonitrile and then dried at room temperature (RT). The appropriate matrix was added, the sample dried at 40° C.

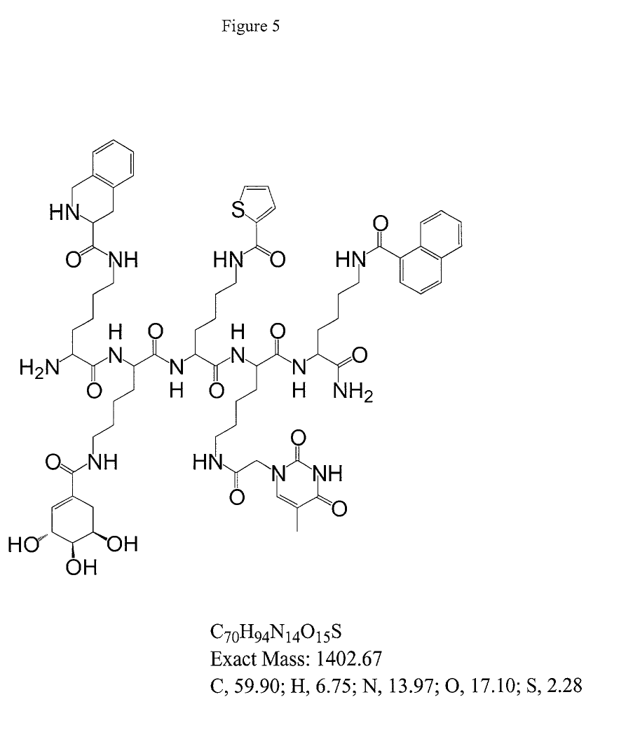

The structure is shown in FIG. 5.

### Example 6

### Derivatization with para-nitrophenyl (ONp) and Succinimide (OSu) Active Esters

High yields of oligomeric N-sec-butylglycyl protecting groups are readily obtained using strong activation during amide-bond formation on the solid-support. The UCP oligomers were completely inert to less activated carboxylic derivatives, such as para-nitrophenyl (ONp) and succinimide (OSu) esters. ONp or OSu esters may be preferred for derivatization because they can be use in large excess while still maintaining selectivity. Nitrophenyl and succinimide esters are readily prepared, and many are commercially available. The inert character of the protecting groups under acylation conditions allows for the chemoselective derivatization of newly liberated primary amino group with nitrophenyl esters. That is, clear chemical selectivity towards the amino-terminus of the protecting groups is employed to distinguish between acylation and deprotection steps.

Boc-Ala-ONp (15.5 mg, 0.05 mmol, 5 mole equivalents) or another preformed ONp or Su ester derivative, is dissolved in anhydrous DMF to a concentration of 0.3 M. The solution is then added to a neutralized resin-bound primary amine (0.1 mmol equivalent) at room temperature and left for 1 h. Depending on the nature or reactivity of the ONp or OSu ester and also the accessibility of the resin-bound primary amine the optimal reaction time may be shorter or longer.

### Example 7

### Synthesis of 2-amino Benzoic Acid Oligomers as Protection Groups

Anthranilic acid, 2-nitrobenzoyl chloride and methyl anthranilate were obtained from the Fluka chemical company. Synthesis carried out in analogous to literature procedure Y. Hamuro, S. J. Geib, and A. D. Hamilton, J. Am. Chem. Soc., 118 (32), pp.7529-7541 (1996). 2-(2-Nitrobenzoylamino)benzoic acid methyl ester was prepared from a solution of methyl anthranilate (30.2 g, 200 mmol) and pyridine (16.6 g, 220 mmol) in dry DCM (200 mL) was cooled in an ice bath with stirring. A solution of 2-nitrobenzoyl chloride in DCM (150 mL) was added dropwise for 15 min to the reaction mixture, and then pyridine (16.6 g) was added to the mixture. The mixture was stirred at room temperature for an additional 16 h. DCM (450 mL) was added to the mixture which was then washed with 1M HCl (500 mL), saturated aqueous NaHCO₃ (200 mL), and brine (200 mL). The organic layer was dried over MgSO₄ and evaporated in vacuo to give crude product (55.3 g, 91%). The crude product was recrystallized from Act (450 mL) and hexane (500 mL) to obtain the desired compound as a pale yellow powder (yield 74%): mp 159.5-160° C. (lit. 146-147° C.).

2-(2-Aminobenzoylamino)benzoic acid methyl ester was prepared from a solution of 2-(2-nitrobenzoylamino)benzoic acid methyl ester (3.85 g, 12.8 mmol) and 10% Pd/C (0.39 g) in DMF (45 mL) under a hydrogen atmosphere and stirred vigorously for 15 h at room temperature. The catalyst was removed by filtration through Celite. DCM (300 mL) was added to the filtrate and then washed with saturated aqueous NaHCO₃ (200 mL) and brine (100 mL). The organic layers were dried over MgSO₄ and evaporated in vacuo to give the desired compound as a red solid (87%). This compound was used without further purification in the next step.

2-(2-nitrobenzoylamino)benzoic acid was prepared from 2-(2-aminobenzoylamino)benzoic acid methyl ester by saponification. A solution of 2-(2-aminobenzoylamino)benzoic acid methyl ester (3 g) in 1M LiOH (15 mL) and THF (25 mL) was stirred vigorously at room temperature for 16 h. The solution was acidified with concentrated HCl and concentrated in vacuo and recrystalized from THF/Hexane (1:1). Yield 82%, mp. 238-239° C. (dec.).

The nitro trimer, 2-(2-(2-Nitrobenzoylamino)benzoylamino)benzoic acid methyl ester, was prepared by a method analogous to the dimer, 2-(2-nitrobenzoylamino)benzoic acid methyl ester. A solution of the 2-nitrobenzoyl chloride (2.21 g, 10.8 mmol) in DCM (50 mL) was added dropwise to a solution of 2-(2-aminobenzoylamino)benzoic acid methyl ester (2.93 g, 10.8 mmol) and pyridine (20.1 g, 60 mmol) in DMF (36 mL) and DCM (36 mL). The reaction mixture was left stirring under an argon atmosphere for 5 days. DCM (400 mL) was added to the mixture was then washed with 1M HCl (200 mL), saturated aqueous NaHCO₃ (200 mL), and brine (200 mL). The organic layer was dried over MgSO₄ and evaporated in vacuo to give crude product. The crude product was recrystallized from Act and hexane three times to obtain the desired compound as thin white needles (54%). mp 168° C. (lit. 155° C.).

2-(2-(2-Aminobenzoylamino)benzoylamino)benzoic acid methyl ester was prepared from a solution of 2-(2-(2-nitrobenzoylamino)benzoylamino)benzoic acid methyl ester (3.41 g, 8.8 mmol) and 10% Pd/C (0.64 g) in DMF (30 mL) under a hydrogen atmosphere and stirred vigorously for 16 h at room temperature. The catalyst was removed by filtration through Celite. DCM (300 mL) was added to the filtrate and then washed with saturated aqueous NaHCO₃ (200 mL) and brine (100 mL). The organic layers were dried over MgSO₄ and evaporated in vacuo to give the desired compound as a yellow solid (87%). This compound was used without further purification in the next step.

2-(2-(2-Aminobenzoylamino)benzoylamino)benzoic acid was prepared from 2-(2-(2-aminobenzoylamino)benzoylamino)benzoic acid methyl ester by saponification. A solution of 2-(2-(2-Aminobenzoylamino)benzoylamino)benzoic acid methyl ester (3 g) in 1M LiOH (15 mL) and THF (25 mL) was stirred vigorously at room temperature for 16 h. The solution was acidified with concentrated HCl, THF was removed in vacuo, the product filtered, washed with water, and dried to a give a give slightly red solid. Yield 85%.

The nitro tetramer, 2-(2-(2-(2-nitrobenzoylamino)benzoylamino)benzoylamino)benzoic acid methyl ester, was prepared by a method analogous to the trimer, 2-(2-(2-aminobenzoylamino)benzoylamino)benzoic acid methyl ester. A solution of the 2-nitrobenzoyl chloride (1.78 g, 9.6 mmol) in DCM (20 mL) was added dropwise to a solution of 2-(2-(2-Aminobenzoylamino)benzoylamino)benzoic acid methyl ester (3.14 g, 8.75 mmol) and pyridine (7.5 g) in DMF (25 mL) and DCM (25 mL). The reaction mixture was left stirring under an argon atmosphere for 3 days. DCM (600 mL) was added to the mixture was then washed with 1M HCl (200 mL), saturated aqueous NaHCO₃ (200 mL), and brine (200 mL). The organic layer was dried over MgSO₄ and evaporated in vacuo to give crude product. The crude product was recrystallized from AcOEt and hexane three times to obtain the desired compound as a white solid (78%). m.p 234-237° C.

2-(2-(2-(2-Aminobenzoylamino)benzoylamino)benzoylamino)benzoic acid was prepared from a solution of 2-(2-(2-(2-nitrobenzoylamino)benzoylamino)benzoylamino)benzoic acid (3.41 g, 8.8 mmol) and 10% Pd/C (0.64 g) in DMF (30 mL) under a hydrogen atmosphere and stirred vigorously for 16 h at room temperature. The catalyst was removed by filtration through Celite. DCM (300 mL) was added to the filtrate and then washed with saturated aqueous NaHCO₃ (200 mL) and brine (100 mL). The organic layers were dried over MgSO₄ and evaporated in vacuo to give the desired compound as a yellow solid (87%). This compound was used without further purification in the next step.

2-(2-(2-(2-nitrobenzoylamino)benzoylamino)benzoylamino)benzoic acid was prepared from 2-(2-(2-(2-aminobenzoylamino)benzoylamino)benzoylamino)benzoic acid methyl ester by saponification. A solution of 2-(2-(2-(2-aminobenzoylamino)benzoylamino)benzoylamino)benzoic acid methyl ester (1.2 g) in 1M LiOH (12 mL) and THF (40 mL) was stirred vigorously at room temperature for 18 h. THF was removed in vacuo, the product filtered, washed with water, and dried to a give white solid. Yield 78%.

Benzyl amine was acylated of in the presence of 2-(2-aminobenzoylamino)benzoic acid methyl ester (UCP-dimer). 2-(2-Aminobenzoylamino)benzoic acid methyl ester (0.089 mmol) and benzyl amine (0.089 mmol) were dissolved in DMF (1 mL). Boc-Ala-OSu (0.132 mmol, 1.5 equiv) and DIPEA (0.265 mmol, 3 equiv) were added, mixed thoroughly, and left at room temperature for 40 min. The reaction was monitored TLC (silica; eluent: ethyl acetate:petroleum spirit (1:3)). After the reaction time, it was found by TLC and ES-MS analysis that the benzyl amine was completely acylated and the starting dimeric UCP-protecting group was unreacted. The presence of the Boc-alanine acylated derivative of the dimeric protecting group was not observed.

### Example 8

### Synthesis of Oligo-N-iso-propylalanyl Protection Groups

Azidoalanine was prepared from the reaction between (S)-(−)-2-brompropionoic acid (BrCH(CH₃)CO2H, Fluka, 99%) and sodium azide (NaN₃, Aldrich 99%). A 1:3 mixture of BrCH(CH₃)CO2H (2 g) and saturated aqueous NaN₃ was stirred continuously in an ice bath for 24 h and subsequently acidified with aqueous HCl (1:1) to pH=5. The product, BrCH(CH₃)CO2H, was then extracted with diethyl ether and dried over anhydrous MgSO₄. Trace amounts of diethyl ether and water were removed under vacuum for 3 days at room temperature. Yield 63%.

Oligo-N-isopropylalanyl protecting group can be prepared on the solid-support, albeit in lower than expected yields due to significant DKP formation with ester type resin linkages. PEGA₁₉₀₀ resin (acryloylated bis(2-aminopropyl)poly(ethylene glycol)/acrylamide copolymer, 0.1 mmol; 0.2 mmol/g, 300-500 μm) was derivatized with [4-(3-hydroxy-3-methyl-butyl)-phenyl]-acetic acid (0.5 mmol) using TBTU (0.48 mmol) and NEM (1 mmol) in DMF (3 mL) for 5 h.

After washing the resin with DMF and DCM, Fmoc-Ala-OH (1 mmol) was loaded onto the resin with MSNT (0.95 mmol) and MeIm (0.75 mmol) in anhydrous DCM (5 mL) for 18 h. The Fmoc group was removed by treatment with 20% piperidine in DMF for 20 min. After washing with DMF and MeOH:DMF:AcOH (9:9:2), a 1:1 mixture of dry acetone in MeOH:DMF:AcOH (9:9:2) was reacted with the resin for 2 h twice. 5 equiv of NaBH₄ in MeOH was added to the resin, stirred for 5 min, and left for another 10 min. After washing with DMF, MeOH, H₂O, MeOH, DMF, THF and DCM, the resin was lyophilized for 16 h.

Azidoalanine chloride was prepared from azidoalanine by refluxing in thionyl chloride (SOCl₂):DCM (1:1) for 3 h. After concentration in vacuo, the azidoalanine chloride was dissolved in 3 mL pyridine:DCM (1:1) and immediately added to the resin. Activated 6 Å molecular sieve were also added. The reaction was left at room temperature under argon for 2 days. After washing the resin with DCM, DMF, MeOH, and DMF, the azide group was reduced with a 0.2M 1,4-dithiothreito (DTT) solution in 10% (v/v) 1,8-diazabicyclo[5.4.0]undec-7-ene (1,5-5) (DBU) DMF at room temperature for 2 h.

After washing with DMF and MeOH:DMF:AcOH (9:9:2), a 1:1 mixture of dry acetone in MeOH:DMF;AcOH (9:9:2) was reacted with the resin for 2 h twice. Five equiv of NaBH₄ in MeOH was added to the resin, stirred for 5 min, and left for another 10 min twice. After washing with DMF, MeOH, H₂O, MeOH, DMF, THF and DCM, the resin was lyophilized for 16 h.

Azidoalanine chloride was prepared from azidoalanine by refluxing in thionyl chloride (SOCl₂):DCM (1:1) for 3 h. After concentration in vacuo, the azidoalanine chloride was dissolved in 3 mL pyridine:DCM (1:1) and immediately added to the resin. Activated 6 Å molecular sieve were also added. The reaction was left at room temperature under argon for 2 days and reduced to the amine with DTT as described above.

After washing with DCM, DMF and MeOH:DMF;AcOH (9:9:2), a 1:1 mixture of dry acetone in MeOH:DMF:AcOH (9:9:2) was reacted with the resin for 2 h twice. 5 equiv of NaBH₄ in MeOH was added to the resin, stirred for 5 min, and left for another 10 min. After each reductive amination step, approximately 30% of the resin was separated and cleaved with TFA at room temperature for 2 h. The product is then extracted in 70% (v/v) acetonitrile/H₂O and filtered and purified by RP-HPLC chromatography.

Alternatively and in general, other N-alkylated oligopeptide protecting groups may be prepared by per-alkylation of linear peptides precursors with e. g. benzyl halides (or other alkylating reagents) which may be substituted with aromatic electron-withdrawing groups, such as NO₂ or Cl. Deprotonation of backbone amides can be achieved on the solid-support with strong bases, such as sodium hydride (NaH) or 2-tert-Butylimino-2-diethylamino-1,3-dimethyl-perhydro-1,3,2-diazaphosphorine (Bemp).

### Example 9

### Synthesis of the N^{α}-(Fmoc-UCP-ₘ₌₁₋₈)-Lysine(N^{ε}-Fmoc-UCPₙ₌₁₋₈)-OH (Where m and n are Different or Identical)

The assembly was performed as in Example 3 on Wang resin (1.01 g, loading: 0.83 mmol/g). The resin was lyophilized for 4 h and then acylated twice with N^{α}-Fmoc-Lys(N^{ε}-Alloc)-OH (2 mmol) or N^{α}-Alloc-Lys(N^{ε}-Fmoc)-OH (2 mmol), Fmoc-1-(mesitylene-2-sulfonyl)-3-nitro-1H-1,2,4-triazole (MSNT, 2mmol) and N-methylimidazole (MeIm, 4 mmol) in dry DCM (10 mL). The resin was washed and Fmoc removed. After thorough washing with DMF, the newly liberated alpha-amine was protected by 4 treatments with trityl chloride (2 mmol) and DIPEA (4 mmol) in DCM 10 mL.

After washing with DCM and lyophilization for 16 h, the N^{α}- or N^{ε}-Alloc group was removed with two treatments of tetrakis(triphenylphosphine)palladium(0) ((PPh₃)₄Pd, 405 mmol), N-ethylmorpholine (NEM)-acetic acid (9.5:10) in DCM for 1 h at room temperature under argon atmosphere. After washing the resin with DCM and DMF, the N^{α}- or N^{ε}-amino group was acylated with N-fluoren-9-ylmethoxycarbonyl-N-sec-butyl-glycine (0.913 mmol, 1.1 equiv) using N,N,N′,N′-tetramethyl-O-(benzotriazol-1-yl)uronium tetrafluoroborate (TBTU) (0.910 mmol)/NEM (2.7 mmol) in DMF for 14 h under an argon atmosphere.

For subsequent couplings the Fmoc group was deprotected with 20% piperidine in DMF for 20 min and following DMF washing N-fluoren-9-ylmethoxycarbonyl-N-sec-butyl-glycine (0.900 mmol) was coupled with bromo-trispyrrolidinophosphonium hexafluorophosphate (PyBroP, 0.899 mmol) and DIPEA (1.8 mmol). This was repeated |n-m| cycles.

The temporary N^{α}- or N^{ε}-trityl group was removed by 3 treatments with 0.1% TFA in DCM for 20 min. Following a DCM wash, the resin was subjected to further cycles of N-fluoren-9-ylmethoxycarbonyl-N-sec-butyl-glycine using bromo-trispyrrolidinophosphonium hexafluorophosphate (PyBroP) and DIPEA (1.1 equiv). A portion of the growing N^{α}-Fmoc-UCPₘ-Lys(N^{ε}-Fmoc-UCP)ₙ-OH oligomer is cleaved off from an aliquot sample of the resin after 1, 3, 5, 7 . . . cycles of N-sec-butylglycine unit coupling.

The building blocks are all purified by HPLC, characterised by MALDI-MS and used to assemble a multifunctional UCP-protected multiple antigen peptide (MAP) carrier on solid support. In this manner all permutations of N^{α}-Fmoc-UCPₘ-Lys(N^{ε}-Fmoc-UCPₙ)-OH can be synthesized

The UCP compound can be used in assembly of MAP molecules as vaccines with different display of the multiple antigens. For example, all possible T-cell determinants and B-cell determinants form a bacterial or viral target protein can be assembled on the same lysine dendrimer. Furthermore, a sequence that target specific importer molecules on macrophages, T-cells or B-cells may be assembled on the same MAP.

### Example 10

### Synthesis of Unichemo Protected MAP Carrier

For the attachment of the MAP to the solid-support a photolabile 4-4-[1-(9H-fluoren-9-ylmethoxycarbonylamino)-ethyl]-2-methoxy-5-nitro-phenoxy-butanoic aminomethyl linker was used. N^{α}-Fmoc-Lys(N^{ε}-Alloc)-OPfp was coupled to the resin and Fmoc group removed. It was coupled with Fmoc-β-alanine and after Fmoc removal N^{α}-Fmoc-UCP₁-Lys(N^{ε}-Fmoc-UCP₂)-OH (1.5 eqv) was coupled using TBTU and N-ethyl morpholine activation.

The Alloc group was removed using the Pd (0) reaction described above and N^{α}-Fmoc-UCP₃-Lys(N^{ε}-Fmoc-UCP₄)-OH was coupled. The Fmoc groups were removed and a unit from the UCP derivatized scaffold was removed by subjection to three treatments of 25% (v/v) phenylisothiocyanate (PITC), 10% (v/v) N-methylpiperidine (NMP) in DMF for 30 min at 55° C.

After the reaction, the resin was washed with DMF then DCM and treated twice with excess neat TFA at 30° C. for 30 min. The resin was treated with N^{α}-Fmoc-UCP₁-Lys(N^{ε}-Fmoc-UCP₂)-OH (1.1 eqv), (TBTU 1.1 eqv) and N-ethyl morpholine (1.3 eqv). Another cycle of UCP cleavage was performed and N^{α}-Fmoc-UCP₃-Lys(N^{ε}-Fmoc-UCP₄)-OH (1.1 eqv) was coupled.

Two more of these cycles were performed to introduce N^{α}-Fmoc-UCP₅-Lys(N^{ε}-Fmoc-UCP₆)-OH and N^{α}-Fmoc-UCP₇-Lys(N^{ε}-Fmoc-UCP₈)-OH. All Fmoc groups were removed. A small fraction of the resin was cleaved by photolysis and the product was characterized by MALDI-MS.

The protected UCP compound is shown in FIG. 6.

### Example 11

### Preparation of Various Antigens

Eight antigenic viral coat Foot and Mouth disease T-cell (VP4 20-35, VP1 135-154, VP1 170-189) B-cell (VP1 39-61, VP1 50-69, VP1 140-160, VP1 197-213) peptides and a T-cell enhancer (sperm whale muoglobin 132-148) were synthesized by conventional peptide synthesis on PEGA resin using a Rink-amide linker. To introduce chemoselective reactive site they were N-terminally derivatised with N-Boc-aminooxoacetyl-N-hydroxysuccinimide ester (2.5 eqv). They were cleaved off the resin with TFA (90%), EDT (3%), Thioanisole (5%) and anisole (2%) and purified by HPLC. The mass was determined by MALDI-MS. These peptide amides were ligated to the UCP-protected MAP sequentially.

### Example 12

### Synthesis of MAP Derivatized with Antigen

The primary amino groups on the MAP were successively liberated with PITC/TFA deprotection cycles: the UCP derivatized scaffold was subjected to three treatments of 25% (v/v) phenylisothiocyanate (PITC), 10% (v/v) N-methylpiperidine (NMP) in DMF for 30 min at 55° C. After the thiourea formation, the resin was washed with DMF then DCM and treated twice with excess neat TFA at 30° C. for 30 min. To introduce a complimentary chemoselective group the liberated primary amine was coupled with glyoxalic acid (1.5 eqv.) activated by TBTU and N-ethyl morpholine in DMF. The resin was carefully washed with DMF and water, and an overnight ligation reaction with the VP4 20-35 peptide derivative (2 eqv) was performed. The resin was carefully washed with DMF, reacted with Fmoc-O-NSu (10 eqv) overnight and washed again with DMF.

The UCP-deprotection cycle was performed, glyoxalic acid was coupled and the first B-cell epitope VP1 39-61 was ligated over night and reacted with Fmoc-O-NSu and washed. This cycle was repeated in the order of ligation: VP1 135-154, VP1 50-69, VP1 170-189, VP1 140-160, sperm whale muoglobin 132-148 and VP1 197-213. The resin was carefully washed with DMF, 20% piperidine in DMF, DMF and water and subjected to photolysis. The product was purified by HPLC and characterized by MALDI-MS.

The final product is shown in FIG. 6.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. A method of forming an intermediate compound for preparing a target compound with different functionality comprising:
(a) preparing two or more protection groups comprising building block units linked together;
(b) forming a protected compound comprising two or more protection groups, wherein at least two of the protection groups contain a different number of building block units;
(b) removing a terminal building block unit of each protection group using one or more chemical, electrochemical, or photolytic reactions; and
(c) consecutively removing an additional building block unit on each remaining protection group.

2. The method of claim 1, wherein the building block units of the protection groups are linked by a C—X—C bond where X is NR, O, S, SiR₂, C≡C, O—SiR₂—O, PR, O—PO—O, O—PO₂—O, CONR, O—CO—O, NR—CO—O, NR—CO—NR, O—S(O₂), an orthoester, an acetal, a ketal or NR—S(O₂); and R is hydrogen, an alkyl, an allyl, an alkene, an alkyne, an aryl, or an alkoxy group.

3. The method of claim 1, wherein the protection group building block units are linked by an amide bond.

4. The method of claim 1, wherein the protection group building block units are alpha, beta or gamma amino acid units.

5. The method of claim 4, wherein the amino acid units are N-substituted with a (C₁ to C₁₀) alkyl or aryl group.

6. The method of claim 5, wherein the amino acid units are N-substituted with a methyl, ethyl, isopropyl, sec-butyl, t-butyl, 3-pentyl, phenyl, benzyl, or halogenated derivatives thereof.

7. The method of claim 4, wherein the amino acid units are unsubstituted or substituted 2-amino benzoic acid or (2-amino-phenyl)-acetic acid.

8. The method of claim 4, wherein the amino acid unit is unsubstituted or substituted glycine, alanine, or alpha amino isobutyric acid.

9. The method of claim 8, wherein the amino acid is N-sec-butyl-glycine.

10. A method of preparing target compounds with different functionality comprising:
(a) preparing a protected template molecule consisting of:
(i) a template molecule having more than one functional group;
(ii) protection groups attached to more than one functional group of the template molecule, the protection groups comprising building block units linked together, wherein
(a′) a first protection group has at least one building block unit; and
(b′) at least one other protection group has more building block units than the first protection group;
(b) removing one or more building block units from each protection group using chemical, electrochemical, or photolytic reactions to form at least one exposed functional group of the template molecule that is not attached to a protection group; and
(c) reacting the exposed functional group of the template molecule with a first target group;
(d) consecutively removing additional building blocks from the protection groups using chemical, electrochemical, or photolytic reactions to form at least one additional exposed functional group of the template molecule that is not attached to a protection group; and
(e) consecutively reacting the additional exposed functional group with an additional target group.

11. The method of claim 10, wherein the building block units of the protection groups are linked by a C—X—C bond where X is NR, O, S, SiR₂, C≡C, O—SiR₂—O, PR, O—PO—O, O—PO₂—O, CONR, O—CO—O, NR—CO—O, NR—CO—NR, O—S(O₂), an orthoester, an acetal, a ketal or NR—S(O₂); and R is hydrogen, an alkyl, an allyl, an alkene, an alkyne, an aryl, or an alkoxy group.

12. The method of claim 10, wherein the protection group building block units are linked by an amide bond.

13. The method of claim 10, wherein the protection groups are oligomers of N-sec-butyl-glycine.

14. The method of claim 10, wherein the template molecule has functional groups selected from the group consisting of an amine, an amide a hydroxyl, a thiol, a carboxylate, or a mixture thereof.

15. The method of claim 10, wherein the template molecule is an oligopeptide, an oligosaccharide or a DNA fragment.

16. The method of claim 10, wherein one of the functional groups of the template molecule is attached to a resin.

17. The method of claim 10, wherein the template is a solid substrate.

18. The method of claim 17, wherein the solid substrate is a glass.

19. The method of claim 17, wherein the solid substrate is a polymer containing functional groups selected from the group consisting of hydroxyl, carboxylate, amino, and combinations thereof.

20. A compound consisting of:
(a) a template molecule having more than one functional group;
(b) protection groups attached to more than one functional group of the template molecule, the protection groups comprising building block units linked together, wherein
(i) a first protection group has at least one building block unit; and
(ii) at least one other protection group has more building block units than the first protection group.

21. The compound of claim 20, wherein the template molecule has functional groups selected from the group consisting of an amine, an amide a hydroxyl, a thiol, a carboxylate, or a mixture thereof.

22. The compound of claim 20, wherein the template molecule is an oligopeptide, an oligosaccharide or a DNA fragment.

23. The compound of claim 20, wherein the protection group are oligomers of N-sec-butyl-glycine.

24. The compound of claim 20, wherein the protection groups are unsubstituted or substituted oligomers of 2-amino benzoic acid.

25. The compound of claim 20, wherein the protection groups are unsubstituted or substituted oligomers of (2-amino-phenyl)-acetic acid.

26. The compound of claim 20, wherein the protection groups are oligomers of N-(1-isopropyl-2-methyl-propylamino)acetic acid.

27. The compound of claim 20, wherein the protection groups are oligomers of N-(1-ethyl-propylamino acid).

28. A compound prepared according to the method of claim 10.

29. A multiple antigen peptide prepared according the method of claim 10.

30. The multiple antigen peptide of claim 29, wherein the template molecule is a peptide chain and the target groups are two or more antigens.

31. The multiple antigen peptide of claim 29, wherein the template molecule is a peptide chain and at least one of the target groups is a T-cell determinant from a human, parasitic, bacterial, or viral protein.

32. The multiple antigen peptide of claim 29, wherein the template is a peptide chain and at least one of the target groups is a B-cell determinant from a human, parasitic, bacterial, or viral protein.

33. A de novo protein prepared according to the method of claim 10.

34. The de novo protein of claim 33, wherein the template is a cyclic peptide and functional secondary structures are attached to form a folded structure.

35. The de novo protein of claim 34, wherein the secondary structure includes a α helix.

36. The de novo protein of claim 34, wherein the secondary structure includes β sheets.

37. The de novo protein of claim 34, wherein the secondary structure contains a catalytic triad.

38. A method of using protection groups to produce microarrays on a solid support comprising:
(a) forming two or more protection groups comprising building block units linked together;
(b) attaching the protection groups to the functional groups of a solid support at a multiple of distinct locations, wherein at least two of the protection group contain a different number of building block units;
(c) removing one or more building block units from each protection group using chemical, electrochemical, or photolytic reactions to form at least one exposed functional group on the solid support;
(d) reacting the exposed functional group of the solid support with a target group;
(e) consecutively removing additional building block units from the protection groups using chemical, electrochemical, or photolytic reactions to form at least one additional exposed functional group on the solid support; and
(f) consecutively reacting the additional exposed functional group of the solid support with additional target groups.

39. The use of claim 38, wherein the target groups are DNA arrays.

40. The use of claim 38, wherein the target groups are oligosaccharides arrays.

41. The use of claim 38, wherein the target groups are protein arrays.

42. The use of claim 38, wherein the target groups are antibody arrays.

43. The use of claim 38, wherein the target groups are useful for biomolecular screening.

44. The use of claim 38, wherein the solid support is a glass.

45. The use of claim 38, wherein the solid support is a polymer containing functional groups selected from the group consisting of hydroxyl, carboxylate, amino, and combinations thereof.

46. The use of claim 38, wherein the solid support is a coating, membrane, plate, particle, or bead.

47. A method for biomolecular screening comprising using microarrays on a solid support according to claim 38.
